(19) United States
(12) Patent Application Publication
(10) Pub. No.: US 2015/0306356 A1
(43) Pub. Date: Oct. 29, 2015

(54) Catheter Insertion Device

(71) Applicant: Sukhjit Gill, Oakbrook, IL (US)

(72) Inventor: Sukhjit Gill, Oakbrook, IL (US)

(21) Appl. No.: 14/675,446

(22) Filed: Mar. 31, 2015

Related U.S. Application Data
(60) Provisional application No. 61/973,132, filed on Mar. 31, 2014.

(51) Int. Cl. A61M 25/09; A61M 25/06

(52) U.S. Cl. CPC A61M 25/09; A61M 25/0606

(57) ABSTRACT

A modular catheter insertion assembly is disclosed. The insertion assembly includes a hub module releasably connected to an insertion module coupled to a hollow needle assembly and a catheter. The hub module is further releasably connected to a guidewire module with a loop housing configured to accommodate a guidewire.

### BACKGROUND

The present disclosure relates generally to the field of medical devices. More specifically, the present invention relates to the field of catheter insertion assembly for use in placement of a catheter into a patient with minimal trauma to the tissues of the patient.

Peripherally inserted central venous catheters (“CVC”) have been utilized by clinicians for several decades. Catheter insertion by the Seldinger technique has been used even longer, primarily for the insertion of subclavian and other chest inserted catheters. The Seldinger technique begins with obtaining access to a blood vessel with a hollow needle. After it is determined that the needle has been inserted into the appropriate blood vessel, a wire is passed through the needle bore into the blood vessel. The wire is often referred to as a “guidewire” since its ultimate purpose is to guide a catheter to a desired site. Once it is determined that a distal end of the guidewire is properly placed within the blood vessel, the needle is removed by backing the needle over the guidewire while leaving the guidewire in place. Proper placement of the guidewire may be verified by fluoroscopy or other imaging methods.

The guidewire is then used to guide a dilator, if needed, into the blood vessel to widen the opening through the skin and subcutaneous tissue. After use, the dilator is removed while the guidewire is still held in place. Multiple dilators may be used, one after the other, until the opening is large enough to receive a catheter introducer. The catheter introducer is a short hollow tube which is placed in the opening. The introducer is sometimes disposed about the largest dilator and inserted along with the dilator. When the dilator is removed the introducer remains. Alternatively, the catheter introducer is inserted subsequent to the removal of the final dilator. With the introducer in place, the catheter is advanced over the guidewire and through the introducer. When catheter insertion is accomplished, the introducer is pulled out of the incision and split according to the manufacturer's usage directions so that it can be removed from around the catheter. The guidewire is removed either prior to or after the removal of the introducer.

One reason why a catheter introducer is necessary is that most catheters are soft and subject to bending and kinking. Inserting a soft and pliable catheter through the tissue of an insertion site and into the vasculature of a patient, even with the use of a guidewire and one or more dilators, is difficult. Such an insertion can result in damage to the catheter, to the patient or both.

### SUMMARY

Modular systems and devices for inserting a catheter are disclosed.

In one aspect of the present disclosure, a modular catheter placement system comprises an insertion module comprising a distal portion and a proximal portion, wherein the proximal portion of the insertion module comprises a first connection port and the distal portion of the insertion portion is coupled to a hollow needle assembly and a catheter The catheter placement system further comprises a guidewire housing module with a loop housing configured to accommodate a guidewire and a second connection port. A hub module is configured to be releasably coupled with the insertion module and the guidewire module via the first connection port and the second connection port; wherein said hub module comprises a central access lumen that is configured to enable the advancement of the guidewire from the guidewire module into a body region via the needle assembly; and wherein the hub module further comprises one more access ports configured to allow a user to access the body region.

In one aspect, the guidewire module further comprises a guidewire advancement mechanism configured to advance or retract the guidewire from the loop housing.

In another aspect, at least one of the access ports is connected to the hub module by an elongated element.

In one aspect, at least one of the connection ports is covered by a membrane. In yet another aspect, the system comprises one or more locking elements configured to prevent the hub module from separating from the first connection port or the second connection port.

In one aspect, at least one of the connection ports comprises an internal portion that is configured to be received by an external portion disposed on the hub module. In another aspect, at least one of the connection ports comprises an external portion that is configured to receive an internal portion disposed on the hub module. Furthermore, the access port is configured to be connected to an intravenous cannula.

In one aspect, the insertion assembly further comprises an insertion portion, wherein the insertion portion comprises a needle and the catheter is attached to at least a portion of the needle. In another aspect, the method comprises separating the hub portion and the guidewire portion.

Other aspects of the invention including corresponding compositions, methods, and systems are described herein.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1A illustrates a first perspective view of one embodiment of an insertion assembly comprising a guidewire housing portion, a hub portion and an insertion portion.

FIG. 1B illustrates a second perspective view of one embodiment of an insertion system comprising a guidewire housing portion, a hub portion and an insertion portion.

FIG. 1C illustrates a second perspective view of one embodiment of an insertion assembly comprising a guidewire housing portion, a hub portion and an insertion portion.

FIG. 2 illustrates one embodiment of a detailed view of the hub portion.

FIG. 3 illustrates one embodiment of a cross-sectional view of the insertion assembly.

### DETAILED DESCRIPTION

While the invention has been disclosed with reference to certain embodiments, it will be understood by those skilled in the art that various changes may be made and equivalents may be substituted without departing from the scope of the invention. In addition, many modifications may be made, to adapt to a particular situation or material, to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The word “exemplary” is used herein to mean “serving as an example, instance, or illustration.” Any implementation described herein as “exemplary” is not necessarily to be construed as advantageous over other implementations.

As used herein, “proximal” describes a location on the invention that is near or toward the patient's skin as the invention is in operation. Conversely, “distal” describes a location on the invention that is farther or away from an anatomical feature than a proximal location as the invention is in operation.

The present disclosure describes devices, systems, and kits of structures and methods for using an insertion assembly to insert a catheter into a patient's body. A broad range of catheter types can be utilized in combination with the structures herein described, such as chronic hemodialysis catheters, central ports, tunneled central catheters, or other catheters normally requiring a catheter introducer.

In some aspects, the present disclosure relates generally to devices, systems, and kits for inserting and accessing a central venous catheter. Central venous catheter is typically inserted by first cleaning the skin, and applying a local anesthetic if required. The location of the vein is then identified by landmarks or with the use of an ultrasound device, which device is optimally functional when used in conjunction with ultrasound scanning gel. Notably, medical sonography is an ultrasound-based diagnostic imaging technique used to externally visualize otherwise internally located muscles, tendons, and organs, including size, structure with real time tomographic images. The scanning gel is superficially applicable to a skin layer as a couplant that provides an acoustic pathway between the transducer and the skin.

In some aspects, the method of placing a central venous catheter comprises inserting or advancing a hollow needle through the skin until blood is aspirated. Then, a guidewire is passed through the needle, and the needle is then removed. A dilating device may be passed over the guide wire to slightly enlarge the tract, and the central line itself is then passed over the guide wire, which is then removed. All the lumens of the line may then be aspirated for the purpose of ensuring proper positioning thereof, and flushed.

Upon the placement of the catheter, the guidewire is removed from the body, and various associated assemblies are detached from the catheter. This procedure may expose the insertion region to infections.

In some aspects, the present disclosure teaches a modular insertion system that is configured to place a catheter, such as a central venous catheter, in the body. The modular design of the present systems facilitate placement of the catheter and enable simple disengagement of the guidewire and other assemblies after the catheter has been placed and thereby minimizing infections at the insertion site.

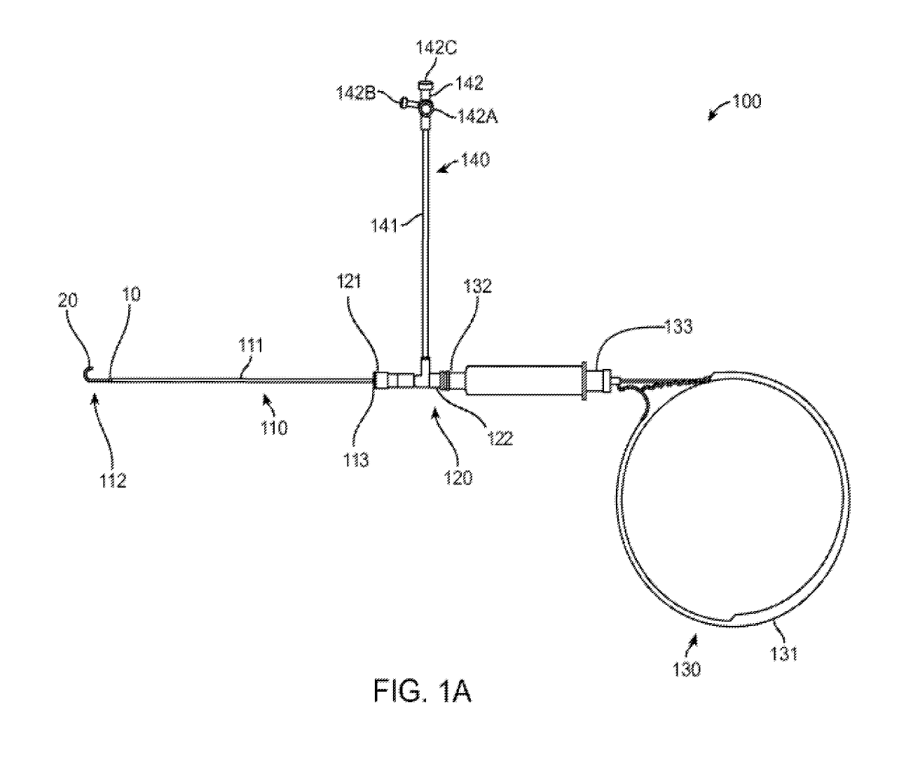

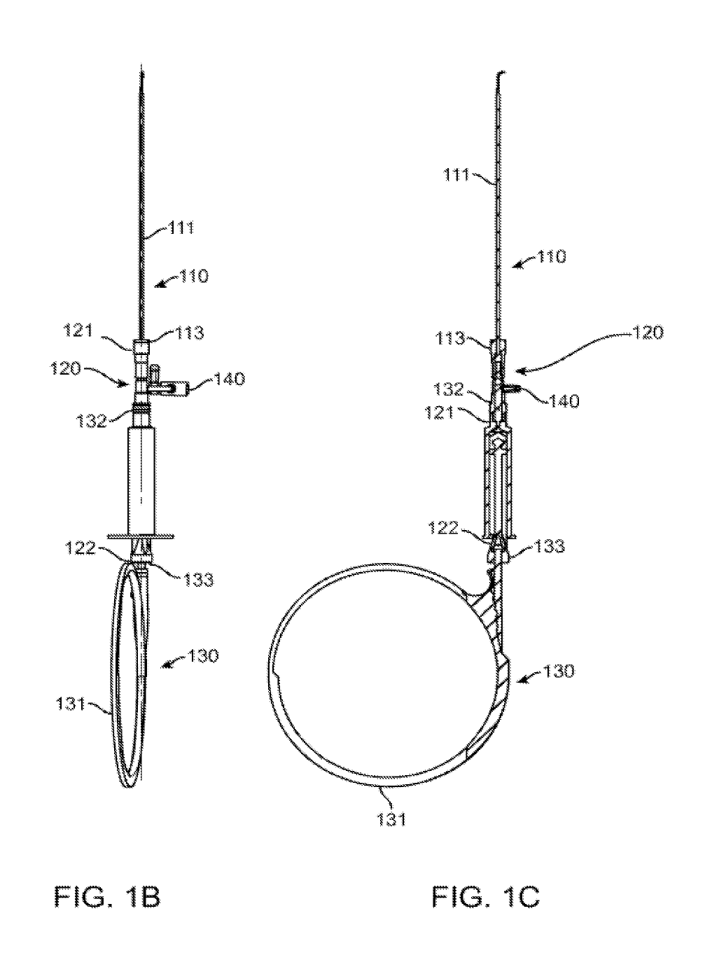

FIGS. 1A-1C show different perspectives of embodiments of the present disclosure. As seen in FIGS. 1A-1C, a modular catheter insertion device 100 comprises an insertion module 110, a hub module 120, and a guidewire module 130. The three modules are configured to be modular where they can be coupled and decoupled by an operator during catheter placement procedure.

In some embodiments, the insertion module 110 comprises an elongate portion 111 that, in one aspect, terminates in a distal portion that is connected to a needle assembly 112. The insertion module 110 optionally comprises a holding section in which a catheter 10 can be press-fitted. The insertion module 110 further comprises a proximal connection section 113 that is configured to be releasably coupled to another module, such as the hub module 120 as described in the present disclosure.

In some embodiments, the needle assembly 112 is a hollow introducer needle coaxially mounted within the catheter 10 and detachably connected to the distal end of the insertion module 110. In one embodiment, the needle is configured with sufficient size such that a guidewire does not completely block the needle, so that blood can traverse through the needle, which will indicate that the tip of the needle is in vessel lumen.

Optionally, in some embodiments, the insertion module 110 further comprises a valve (not shown) that is housed within an insertion lumen (not shown) disposed longitudinally within the insertion module 110. In some aspects, the valve substantially prevents all fluid flow through the insertion module 110 in at least one direction, e.g., from the atmosphere to inside of the patient, below a first pressure threshold. The catheter 10 is in fluid communication with the insertion module 110 such that fluid flow through the lumens of the catheter 10 is controlled by the valve. As a result, the valve substantially prevents all air from entering the catheter 10, but can permit fluid flow into the catheter. Alternatively, the valve may be disposed within the hub module 120.

In some aspects, the insertion lumen of the insertion module 110 functions as a conduit that extends from the proximal connection section 113 to the needle assembly 112. The lumen is configured to enable a guidewire to traverse the insertion module 110 via the connection section 113, past the needle assembly 112, and into the body.

The guidewire module 130 comprises a guidewire housing unit 131, a distal connection section 132, and a guidewire advancement mechanism 133. The guidewire housing 131, in one embodiment, is exemplarily configured as a loop housing. In such embodiment, a guidewire 20 may be wound around the loop such that guidewire of various sizes may be housed within the housing unit 131. Particularly, a guidewire of greater length than the capacity of systems known in the art may be used in the procedure, which may improve the accuracy and the ease of insertion.

In some aspects, the guidewire advancement mechanism 133 is configured to manipulate the guidewire 20. In some aspects, the guidewire advancement mechanism 133 comprises a pusher, wherein the pusher comprises an inner portion that is configured to be slidably moveable within an outer portion of the guidewire module 130. In some aspects, a portion of the pusher is connected to the guidewire 20. In one embodiment, the connection may be achieved, for example, by adhesive, welding, interference fit or press fit. Optionally, the pusher comprises a handle that facilitates the user's manipulation of the guidewire 20, such as the advancement of the guidewire 20. Furthermore, the pusher comprises one or more locking elements configured to halt the advancement of the guidewire 20 when an appropriate degree of advancement has been achieved.

In one embodiment, the guidewire advancement mechanism 133 is configured to be capable of continuously advancing the guidewire 20 from the housing unit 131 distally towards the insertion module 110. In one embodiment, the advancement mechanism 133 is configured to be slidably movable proximally along a portion of a guidewire, then coupling or locking against the guidewire, and advanced along with the guidewire distally towards the insertion portion 110, thereby advancing the guidewire. After advancing a portion of the guidewire, the advancement mechanism 133 is unlocked from the guidewire 20, thus enabling the advancement mechanism 133 to be moved proximally such that another portion of the guidewire 20 may be locked or coupled with the advancement mechanism 133 to continuously advance the guidewire from the housing unit 131 towards the insertion module 110.

In some aspects, the guidewire 20 is configured with sufficient length such that it is capable of extending the entire length of the system 100 where all three modules are connected. In one embodiment, the length of the guidewire 20 is configured to extend at least twice the length of the system 100. It is noted that a longer guidewire 20 may improve maneuverability and improve the ease of catheter placement.

In some embodiments, the guidewire 20 has a stiffness greater than the stiffness of the catheter and therefore stiffens the catheter such that the catheter may be inserted into a patient without buckling, even without using a catheter introducer. This minimizes insertion trauma. A thermoplastic polyurethane resin, e.g., ISOPLAST manufactured by Dow Chemical Company of Midland, Mich., is an example of a material of appropriate characteristics to impart the proper stiffness to the guidewire.

In one embodiment, the hub module 120 is configured to be releasably coupled with the insertion module 110 via a first connection port 121 to the proximal connection section 113 of the insertion module 110. The hub module 120 is further configured to be releasably coupled with the guidewire module 130 via a second connection port 122 to the distal connection section 132 of the guidewire module 130.

The hub module 120 comprises at least one access lumen disposed longitudinally within the hub module 120 that is configured to enable the advancement of the guidewire 20 from the guidewire module 130 into a body region via the insertion module 110 and the needle assembly 112 and/or the catheter 10. The access lumen is configured to align with the insertion lumen of the insertion module 110 such that a continuous pathway is formed from the second connection port 122 which connects to the guidewire module 130, and to the needle assembly 112. This continuous pathway enables a guidewire to traverse from the guidewire module 130 into the body region through the needle assembly while traversing the hub module 120.

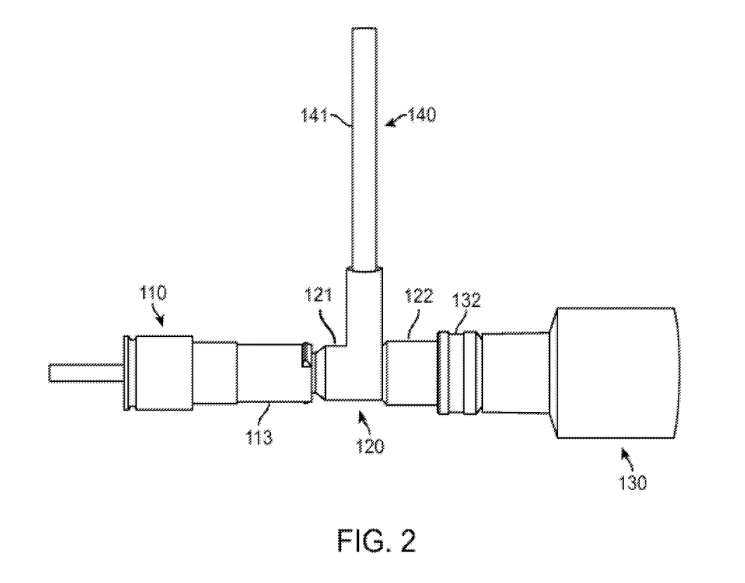

The hub module 120 further comprises one or more access ports 140 connected to the access lumen and configured to allow a user to access a body region. As seen in FIGS. 1A and 1n greater detail in FIG. 2, in some embodiments, the hub module 120 is configured with at least one branching side access port 140. The branching side access port 140 comprises an elongate portion 141, one or more lumens therein that extend to an access portion 142 away from the main body of the hub module 120. The access portion 142 may comprise multiple additional ports 142A, 142B, and 142C. In some aspects, the multiple ports 142A, 142B, 142C enable controlled and individualized access to the body region, where each access port may be dedicated for a specific function.

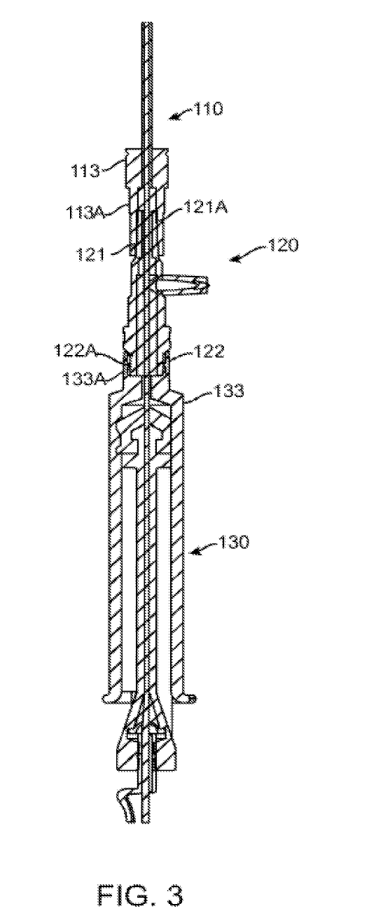

Referring now to FIG. 3, a cross-sectional view of the system is shown with the guidewire module 130 connected to the hub module 120 which is further connected to the insertion module 110. As seen in FIG. 3, in some aspects, the coupling between the insertion module 110 and the hub module 120 is achieved by inserting an internal section 121A of the first connection port 121 of the hub module 120 into an external section 113A of the connection section 113 of the insertion module 110. Similarly, in some aspects, the coupling between the guidewire module 130 and the hub module 120 is achieved by inserting an internal section 122A of the second connection port 122 of the hub module 120 into an external section 133A of the connection section 133 of the insertion module 130.

In some aspects, the modular configurations of the present embodiments is further supported by the standardization of the connection ports and the connection sections. Specifically, the internal/external sections of the connection ports and the connection sections of the different modules may be configured with a standard dimensions such that various modules can be inserted interchangeably.

Alternatively, the external/internal section configurations may be reversed, wherein the internal section is a part of the connection sections of either the insertion module 110 or the guidewire module 130, or both. Correspondingly, one or more of the corresponding external portions are disposed on the hub portion 120.

By inserting the internal segment into the external segment, connections between the various portions are achieved. It is noted, that the connection can be severed by disengaging the internal portion and the external portion.

The external segments, in some aspects, comprise elements that facilitate a user to easily grip the various portions, such as one or more indentations and/or one or more rims. In some embodiments, the connection ports and/or connection sections further comprise locking mechanisms to prevent the separation of the modules. In one embodiment, the locking element is a locking clip that is configured to be received in groove.

It is noted that in some aspects, the openings of the internal section or the external section may be covered by a membrane to prevent contaminants from entering into the portions when the openings are exposed. In one embodiment, the membrane is configured to be penetrable by the guidewire such that the guidewire operation to access the body is minimally impacted.

In one exemplary operation of some embodiments of the present system, the three modular modules are initially connected. Using known methods, the needle assembly is passed through the skin, subcutaneous tissue and vessel wall of a patient. The guidewire is then advanced from the guidewire housing of the guidewire portion through the lumens of the hub portion and the insertion portion, where it is then passed through the needle assembly by manipulating the guidewire using the guidewire advancement mechanism. For example, the user may advance a pusher element to place the guidewire at a desired position within the patient.

Thereafter the catheter is configured to be guided by guidewire through the skin, subcutaneous tissue and vessel wall of the patient and into the vasculature. The stiffness of the guidewire allows the soft, flexible catheter to pass through the subcutaneous tissue without the need for a catheter introducer. Once it has been confirmed that the catheter is in place, the guidewire may be retracted and the guidewire portion can be disconnected from the hub portion by separating the connection portions of the guidewire portion and the hub portion.

It is noted that various devices may be connected to the connection ports of the hub portion to improve or facilitate fluid or drug administration or fluid retrieval from a patient's blood vessels. Additionally, it is contemplated that additional guidewires or devices may be inserted via the connection ports as needed.

The components of the presently disclosed devices and assemblies may be formed from a variety of different materials known in the art. For example, the sheath may be fabricated of polyurethanes or silicone and the needle may be fabricated of stainless steel, titanium, as well as polymers. Additionally, other similar biocompatible materials are also envisioned.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A modular catheter placement system, comprising:
an insertion module comprising a distal portion and a proximal portion, wherein the proximal portion of the insertion module comprises a first connection port and the distal portion of the insertion module is coupled to a hollow needle assembly and a catheter;
a guidewire module comprising a distal portion and a proximal portion wherein the distal portion of the guidewire module comprises a second connection port and the proximal portion comprises a loop housing configured to accommodate a guidewire, and
a hub module that is configured to be releasably coupled with the insertion module and the guidewire module via the first connection port and the second connection port; wherein said hub module is configured to enable the advancement of the guidewire from the guidewire module into a body region via the needle assembly and the catheter; and wherein the hub module further comprises one more access ports configured to allow a user to access the body region.

2. The system of claim 1, wherein the guidewire module further comprises a guidewire advancement mechanism configured to advance or retract the guidewire from the loop housing.

3. The system of claim 1, wherein at least one of the access ports is connected to the hub module by an elongated element.

4. The system of claim 1, wherein at least one of the connection ports is covered by a membrane.

5. The system of claim 1, further comprising one or more locking elements configured to prevent the guidewire module from separating from the first connection port or the second connection port.

6. The system of claim 1, wherein at least one of the connection ports comprises an internal portion that is configured to be received by an external portion disposed on the hub portion.

7. The system of claim 1, wherein at least one of the connection ports comprises an external portion that is configured to receive an internal portion disposed on the hub module.

8. The system of claim 1, wherein the access port is configured to connect to an intravenous cannula.

9. The system of claim 1, wherein the access port is configured to mate with a port of an intravenous device via a luer lock.

10. A method for inserting a catheter into a body region, said method comprising the steps of:
attaching the proximal end of the catheter to a modular insertion assembly comprising:
a hub module comprising a proximal end, a distal end, and a lumen through the hub module accessible via one or more access ports; wherein the hub module is connected to an insertion module and a guidewire module, wherein the insertion module is configured to be connected to a needle assembly and guidewire module comprises a circular guidewire housing configured to retain a guidewire;
inserting a portion of the guidewire from the insertion assembly into a body lumen of a patient;
advancing the distal tip of the catheter over the guidewire to a desired location in the body of the patient; and
removing the guidewire from the insertion assembly.

11. The method of claim 10, wherein the insertion assembly further comprises an insertion portion; wherein the insertion portion comprises a needle and the catheter is attached to at least a portion of the needle.

12. The method of claim 10, further comprising separating the hub portion and the guidewire housing portion.

13. The method of claim 10, further comprising accessing the catheter via the access ports.
